SEQUENCE LISTING (22 sequences)

SEQ ID NO: 1 - protein, 31 aa, Artificial Sequence
  Synthetic
DAVDCALDPLSETKCTLKSFTVEKGIYQTSN

SEQ ID NO: 2 - protein, 75 aa, Artificial Sequence
  Synthetic
VCGPKKSTNLVKNKCVNFNFNGLTGTGVLTESNKKFLPFQQFGRDIADTTDAVRDPQTLEILDITPCSFGGVSVI

SEQ ID NO: 3 - protein, 40 aa, Artificial Sequence
  Synthetic
GTNTSNQVAVLYQDVNCTEVPVAIHADQLTPTWRVYSTGS

SEQ ID NO: 4 - protein, 18 aa, Artificial Sequence
  Synthetic
FSQILPDPSKPSKRSFIE

SEQ ID NO: 5 - protein, 22 aa, Artificial Sequence
  Synthetic
FGAGAALQIPFAMQMAYRFNGI

SEQ ID NO: 6 - protein, 21 aa, Artificial Sequence
  Synthetic
RPQGLPNNTASWFTALTQHGK

SEQ ID NO: 7 - protein, 20 aa, Artificial Sequence
  Synthetic
NNNAATVLQLPQGTTLPKGF

SEQ ID NO: 8 - protein, 47 aa, Artificial Sequence
  Synthetic
NKHIDAYKTFPPTEPKKDKKKKTDEAQPLPQRQKKQPTVTLLPAADM

SEQ ID NO: 9 - protein, 24 aa, Artificial Sequence
  Synthetic
MADSNGTITVEELKKLLEQWNLVI

SEQ ID NO: 10 - protein, 20 aa, Artificial Sequence
  Synthetic
PLLESELVIGAVILRGHLRI

SEQ ID NO: 11 - protein, 42 aa, Artificial Sequence
  Synthetic
MSDNGPQNQRNAPRITFGGPSDSTGSNQNGERSGARSKQRRP

SEQ ID NO: 12 - protein, 42 aa, Artificial Sequence
  Synthetic
QGLPNNTASWFTALTQHGKEDLKFPRGQGVPINTNSSPDDQI

SEQ ID NO: 13 - protein, 42 aa, Artificial Sequence
  Synthetic
GYYRRATRRIRGGDGKMKDLSPRWYFYYLGTGPEAGLPYGAN

SEQ ID NO: 14 - protein, 45 aa, Artificial Sequence
  Synthetic
KDGIIWVATEGALNTPKDHIGTRNPANNAAIVLQLPQGTTLPKKK

SEQ ID NO: 15 - protein, 42 aa, Artificial Sequence
  Synthetic
KGFYAEGSRGGSQASSRSSSRSRNSSRNSTPGSSRGTSPARM

SEQ ID NO: 16 - protein, 42 aa, Artificial Sequence
  Synthetic
AGNGGDAALALLLLDRLNQLESKMSGKGQQQQGQTVTKKSAA

SEQ ID NO: 17 - protein, 42 aa, Artificial Sequence
  Synthetic
EASKKPRQKRTATKAYNVTQAFGRRGPEQTQGNFGDQELIRQ

SEQ ID NO: 18 - protein, 45 aa, Artificial Sequence
  Synthetic
GTDYKHWPQIAQFAPSASAFFGMSRIGMEVTPSGTWLTYTGAKKK

SEQ ID NO: 19 - protein, 42 aa, Artificial Sequence
  Synthetic
IKLDDKDPNFKDQVILLNKHIDAYKTFPPTEPKKDKKKKADE

SEQ ID NO: 20 - protein, 41 aa, Artificial Sequence
  Synthetic
TQALPQRQKKQQTVTLLPAADLDDFSKQLQQSMSSADSTQA

SEQ ID NO: 21 - protein, 69 aa, Artificial Sequence
  Synthetic
SNNLDSKVGGNYNYLYRLFRKSNLKPFERDISTEIYQAGSTPCNGVEGFNCYFPLQSYGFQPTNGVGYQ

SEQ ID NO: 22 - protein, 26 aa, Artificial Sequence
  Synthetic
KKDKKKKADETQALPQRQKKQQTVTL

(19) United States
(12) Patent Application Publication
(10) Pub. No.: US 2024/0310362 A1
(43) Pub. Date: Sep. 19, 2024

(54) METHOD TO IDENTIFY INDIVIDUALS WITH T CELL IMMUNITY TO SPECIFIC INFECTIOUS AGENTS

(71) Applicant: Quidel Corporation, San Diego, CA (US)

(72) Inventors: Richard L. Egan, Sparks, NV (US); Jason J. Sun, San Diego, CA (US); Werner Kroll, Wayland, MA (US)

(21) Appl. No.: 18/571,173

(22) PCT Filed: Jun. 15, 2022
(86) PCT No.: PCT/US2022/033637

§ 371 Date: Dec. 15, 2023

Related U.S. Application Data
(60) Provisional application No. 63/211,398, filed on Jun. 16, 2021.

(51) Int. Cl. G01N 33/50; G01N 21/64

(52) U.S. Cl. CPC G01N 33/505; G01N 21/6428; G01N 2021/6439; G01N 2333/165

(57) ABSTRACT

Methods, devices and kits for rapid identification of individuals previously infected with, or vaccinated for, a bacterial or viral pathogen, such as SARS CoV-2 virus. Pathogen specific memory T cells, such as SARS CoV-2 specific memory T cells, provide indication of prior infection, if detected. Memory T cells are assayed by exposure to specific bacterial and/or viral antigens and then tested for activation by analysis of nucleotide content.

### CROSS-REFERENCE TO RELATED APPLICATIONS

This application claims the benefit of U.S. Provisional Application No. 63/211,398, filed Jun. 16, 2021, which is incorporated by reference herein.

### SEQUENCE LISTING

A “Sequence Listing” is submitted with this application in the form of a text file, created Jun. 14, 2022, and named “0418961281SEQ.txt” (9,816 bytes), the contents of which are incorporated herein by reference in their entirety.

### TECHNICAL FIELD

The subject matter described herein relates to a method that can quickly identify individuals that were previously infected with a bacterial and/or viral pathogen, such as SARS CoV-2 virus. Methods provided herein detect antigen specific memory T cells as the indicator for past infection, rather than the presence of pathogen specific antibodies.

### BACKGROUND

Memory T cells are an important part of the immune response to infectious pathogens that play several roles in defending individuals against bacterial and viral infections. Memory T cells are found in certain tissues and body fluids, such as bone marrow, thymus tissue and blood, and contain antibody-like receptors on their surfaces. When memory T cell surface receptors come in contact with pathogenic antigens, such as protein or peptide based antigens, the cells become “activated” wherein they shed immune effector proteins and begin to replicate.

Each unique subpopulation of memory T cells contains a different antibody-like surface receptor that is specific for a unique foreign antigen. When a memory T cell encounters its corresponding antigen, the antigen is bound by the antigen specific cell surface receptors. Upon antigen-receptor binding, memory T cells undergo a transformation wherein they begin excreting immune effector proteins, such as cytokines, and commence with rapid cell division. This transformation serves to expand the population of memory T cells primed to kill cells expressing the specific foreign antigen.

A critical aspect of memory T cell transformation in response to antigen detection and binding, is production of mRNA for effector protein, such as cytokines, expression, and mRNA for cell division, which requires replication of the full genome. This rapid and extensive alteration in the cells physiologic state is amendable to particular detection methods related to identifying the increased nucleic acid content present in activated memory T cells.

Severe acute respiratory syndrome coronavirus 2 (SARS CoV-2) is the virus strain that causes coronavirus disease 2019 (COVID-19), a respiratory illness. It is colloquially known as the coronavirus, and was previously referred to by its provisional name 2019 novel coronavirus (2019-nCoV). SARS CoV-2 is a positive-sense single-stranded RNA virus. It is contagious in humans, and the World Health Organization designated the 2019 pandemic of COVID-19 a Public Health Emergency of International Concern.

Like other known coronaviruses, SARS CoV-2 is an enveloped virus containing three outer structural proteins, namely the membrane (M), envelope (E), and spike (S) proteins. The nucleocapsid (N) protein together with the viral RNA genome presumably form a helical core located within the viral envelope. The SARS CoV-2 nucleocapsid (N) protein is a 423 amino acid, predicted phospho-protein of 46 kDa that shares little homology with other members of the coronavirus family. SARS CoV-2 uses its spike glycoprotein (S), a main target for neutralization antibody, to bind its receptor, and mediate membrane fusion and virus entry. Each monomer of trimeric S protein is about 180 kDa, and contains two subunits, S1 and S2, mediating attachment and membrane fusion, respectively.

There remains a need for methods, devices and kits that specifically and sensitively identify memory T cells expressing surface receptors specific for particular pathogens of interest, such as the SARS CoV-2 virus. Identification of such memory T cells provides valuable information related to whether an individual has experienced prior exposure (via natural exposure or vaccination) to, and/or infection with, the pathogen of interest. For example, exposure to, infection with, and/or vaccination for particular bacteria and viruses, such as the SARS CoV-2 virus may generate memory T cells specific for the particular pathogen.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

The technology disclosed herein is related to methods, devices and kits for detection of memory T cells that are activated after exposure to specific bacterial and/or viral pathogenic antigens, such as SARS CoV-2 viral antigens. The technology exposes memory T cells from a subject to specific bacterial and/or vial antigens, such as SARS CoV-2 viral antigens. The exposed memory T cells are then assayed for nucleotide content wherein an increased nucleotide content, such as increased RNA and/or DNA content, is indicative of activated memory T cells, as compared to inactive counterparts. Analysis of the cells with a reagent that detects total nucleotide quantity, such as an RNA and/or DNA dye, including fluorescent dyes, allows a practitioner to ascertain whether the memory T cells have increased nucleotide content indicating that cells have encountered their specific pathogenic antigen.

In one aspect, the technology described herein provides a method to determine presence or absence of infectious agent-specific T cells in a sample from a subject. In another aspect, a method to ascertain prior exposure to, or vaccination of a subject for, an infectious agent is provided. In some embodiments, the method comprises exposing a biological sample comprising memory T-cells from the subject to one or more peptide antigens specific for the infectious agent. In some embodiments, the method further comprises contacting the exposed memory T-cells with an indicator compound that associates with RNA, DNA, or both. In some aspects, the method further comprises analyzing the memory T-cells for indicator compound.

In one aspect, the technology described herein provides a method to identify SARS CoV-2-specific T cells in a sample from a subject. In another aspect, a method to ascertain prior exposure to, or vaccination of a subject for, SARS CoV-2 virus is provided.

In some embodiments, the method comprises exposing a biological sample comprising memory T-cells from the subject to one or more peptide antigens specific for SARS CoV-2. In some embodiments, the method further comprises contacting the exposed memory T-cells with an indicator compound that associates with RNA, DNA, or both. In some aspects, the method further comprises analyzing the memory T-cells for indicator compound.

In some embodiments, memory T-cells are exposed to the peptides specific for SARS CoV-2 and contacted with the indicator compound simultaneously. In some embodiments, memory T-cells are exposed to the peptides specific for SARS CoV-2 and contacted with the indicator compound sequentially.

In some aspects, the biological sample is a blood sample and/or a fraction of a blood sample. In some embodiments, the fraction of the blood sample is the buffy coat fraction or peripheral blood mononuclear cells (PBMCs) or a mixture of buffy coat fraction and PBMCs.

In some aspects, exposing the biological sample to one or more peptide antigens specific for SARS CoV-2 comprises exposing to a solution comprising the one or more peptide antigens, the indicator compound and one or more of a buffer, an energy source for the cells, and a balanced salt solution, thereby simultaneously with said exposing, contacting the T cells with the indicator compound.

In some aspects, the method further comprises exposing a second biological sample comprising memory T-cells to a control reagent that (i) lacks the one or more peptide antigens specific for SARS CoV-2 and (ii) comprises a control indicator compound that associates with RNA, DNA, or both, to thereby generate a control sample. In some embodiments, the second biological sample is from a subject, and wherein the biological sample is from the same subject or wherein the second biological sample is a portion of the biological sample. In some embodiments, the indicator compound and the control indicator compound are the same. In other embodiments, analyzing comprises measuring signal of indicator compound associated with memory T-cells in the biological sample and measuring signal of control indicator compound associated with memory T-cells in the control sample. In some embodiments, analyzing comprises measuring signal of indicator compound associated with RNA in the biological sample and measuring signal of control indicator compound associated with RNA in the second biological sample. In other embodiments, analyzing comprises measuring an RNA signal based on signal of indicator compound associated with RNA, measuring a DNA signal based on signal of indicator compound associated with DNA, and determining a ratio of RNA signal to DNA signal or of DNA signal to RNA signal. In some embodiments, the indicator compound is a fluorescent dye that selectively stains RNA.

In another embodiment, the exposed memory T-cells are contacted with a first indicator compound that selectively stains RNA or DNA and with a second indicator compound that non-specifically stains RNA and DNA.

In another aspect, the indicator compound has an excitation between about 330-360 nm and an emission at greater than between about 500-600 nm.

In some aspects, the memory T-cells are CD4+ and/or CD8+ T cells.

In some aspects, the method of any preceding claim, further comprises incubating the biological sample for a period of time, such as about 10-60 minutes or about 10-30 minutes. In some embodiments, the incubating is performed after the sample is exposed to peptide antigens and contacted with indicator, but before analyzing. In some embodiments, the sample is incubated at a temperature of between about 25-40° C.

In another aspect, the one or more peptide antigens specific for SARS CoV-2 comprises between 2-20 peptide antigens specific for SARS CoV-2 or between 3-15 peptide antigens specific for SARS CoV-2. In some embodiments, the one or more peptide antigens specific for SARS CoV-2 comprise one or more of the peptides identified as SEQ ID NO: 1-SEQ ID NO: 12. In some embodiments, the exposing step further comprises exposing the biological sample to one or more peptide antigens non-specific for SARS CoV-2.

In another aspect, the methods provided herein ascertain prior exposure of a subject to an infectious pathogen. In some embodiments, the method comprises exposing a biological sample comprising memory T-cells from the subject to one or more peptide antigens specific for the infectious pathogen. In some embodiments, the method further comprises contacting the exposed memory T-cells with an indicator compound that associates with RNA, DNA, or both. In some aspects, the method further comprises analyzing the memory T-cells for indicator compound.

In some embodiments, memory T-cells are exposed to the peptides specific for the infectious pathogen and contacted with the indicator compound simultaneously. In some embodiments, memory T-cells are exposed to the peptides specific for the infectious pathogen and contacted with the indicator compound sequentially.

In some embodiments, the infectious agent or infectious pathogen is a viral pathogen, such as a respiratory syncytial virus or human coronavirus. In other embodiments, the pathogen is a bacterial pathogen, such as a Borrelia pathogen for Lyme disease.

In addition to the exemplary aspects and embodiments described above, further aspects and embodiments will become apparent by reference to the drawings and by study of the following descriptions.

Additional embodiments of the present methods and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE SEQUENCES

TABLE 1

| SARS CoV-2 | | |
|---|---|---|
| Antigen Peptide | SEQUENCE | SEQ ID NO |
| #1 S (287-317) | DAVDCALDPLSETKCTLKSFTVEKGIYQTSN | 1 |
| #2 S (524-598) | VCGPKKSTNLVKNKCVNFNFNGLTGTGVLTE | 2 |
| | SNKKFLPFQQF | |
| | GRDIADTTDAVRDPQTLEILDITPCSFGGVSVI | |
| #3 S (601-640) | GTNTSNQVAVLYQDVNCTEVPVAIHADQLTP | 3 |
| | TWRVYSTGS | |
| #4 S (802-819) | FSQILPDPSKPSKRSFIE | 4 |
| #5 S (888-909) | FGAGAALQIPFAMQMAYRENGI | 5 |
| #6 N (42-62) | RPQGLPNNTASWFTALTQHGK | 6 |
| #7 N (153-172) | NNNAATVLQLPQGTTLPKGF | 7 |
| #8 N (355-401) | NKHIDAYKTFPPTEPKKDKKKKTDEAQPLPQR | 8 |
| | QKKQPTVTLLPAADM | |
| #9 M (1-24) | MADSNGTITVEELKKLLEQWNLVI | 9 |
| #10 M (132-151) | PLLESELVIGAVILRGHLRI | 10 |
| #11 N Peptide 1 | MSDNGPQNQRNAPRITFGG | 11 |
| | PSDSTGSNQNGERSGARS KQRRP | |
| #12 N Peptide 2 | QGLPNNTASWFTALTQHGK | 12 |
| | EDLKFPRGQGVPINTNSSP DDQI | |
| #13 N Peptide 3 | GYYRRATRRIRGGDGKMKD | 13 |
| | LSPRWYFYYLGTGPEAGLP YGAN | |
| #14 N Peptide 4 | KDGIIWVATEGALNTPKDHI | 14 |
| | GTRNPANNAAIVLQLPQGTT LPKKK | |
| #15 N Peptide 5 | KGFYAEGSRGGSQASSRSS | 15 |
| | SRSRNSSRNSTPGSSRGTS PARM | |
| #16 N Peptide 6 | AGNGGDAALALLLLDRLNQ | 16 |
| | LESKMSGKGQQQQGQTVT KKSAA | |
| #17 N Peptide 7 | EASKKPRQKRTATKAYNVT | 17 |
| | QAFGRRGPEQTQGNFGDQ ELIRQ | |
| #18 N Peptide 8 | GTDYKHWPQIAQFAPSASA | 18 |
| | FFGMSRIGMEVTPSGTWLT YTGAKKK | |
| #19 N Peptide 9 | IKLDDKDPNFKDQVILLNKHI | 19 |
| | DAYKTFPPTEPKKDKKKKA DE | |
| #20 Peptide 10 | TQALPQRQKKQQTVTLLPA | 20 |
| | ADLDDFSKQLQQSMSSADS TQA | |
| #21 S (437-506) | SNNLDSKVGG NYNYLYRLFR KSNLKPFERD | 21 |
| | ISTEIYQAGS TPCNGVEGEN CYFPLQSYGF | |
| | QPTNGVGYQ | |
| #22 N (370-395) | KKDKKKKADETQALPQRQKKQQTVTL | 22 |

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The term “about”, particularly in reference to a given quantity, is meant to encompass deviations of plus or minus five percent.

The compositions of the present disclosure can comprise, consist essentially of, or consist of, the components disclosed.

All percentages, parts and ratios are based upon the total weight of the topical compositions and all measurements made are at about 25° C., unless otherwise specified.

“Sample” is any material to be tested for the presence a particular memory T cell of interest. Preferably, a sample is a fluid sample, preferably a liquid sample. Examples of liquid samples that may be tested using a test device include bodily fluids including blood, serum, plasma, saliva, urine, ocular fluid, semen, sputum, nasal discharge and spinal fluid. For example, a sample for testing on a disclosed device may comprise liquid serum or plasma from a venous blood source where the serum or plasma has been separated from whole blood by centrifugation. In other cases, a sample may be liquid plasma from a finger prick that has been separated from whole blood by a blood-plasma separator. Other sample examples include liquid plasm from a finger prick that has been separated from whole blood by the lateral flow device. In some embodiments, a sample comprises the band that forms, upon centrifugation of whole blood, between red blood cells and plasma. In some instances, this band, also known as the “buffy coat”, comprises the lymphocytes from the whole blood and can be used as the sample for analysis for the presence of particular population of memory T cells.

“Peptide antigen” refers to a protein or peptide that binds to specific receptors present on the cell surface of a particular population of memory T cells. Peptide sequences related to the present disclosure may comprise antigenic peptide or protein from any pathogen of interest, such as a bacterial or viral pathogen. In certain embodiments, the peptide antigens comprise SARS CoV-2 peptide antigens as provided in Table 1. For example, peptide antigens include SARS CoV-2 proteins, peptides, such as SARS CoV-2 membrane (M), envelope (E), spike (S, including S1 and S2 subunits), and nucleocapsid (N) proteins. The nucleocapsid (N) protein together with the viral RNA genome, presumably form a helical core located within the viral envelope. The SARS CoV-2 nucleocapsid (N) protein is a 423 amino-acid, predicted phospho-protein of 46 kDa that shares little homology with other members of the coronavirus family. SARS CoV-2 uses its spike glycoprotein (S), a main target for neutralization antibody, to bind its receptor, and mediate membrane fusion and virus entry. Each monomer of trimeric S protein is about 180 kDa, and contains two subunits, S1 and S2, mediating attachment and membrane fusion, respectively.

In some embodiments, SARS CoV-2 peptide antigens include full length N-protein, and specific epitopes of full length N-protein. Proteins and peptides may be selected as reaction partners based on sequences and/or immunogenicity analysis represented by respective peptides. Peptides represented by a SARS CoV-2 N-protein epitope map based on segmenting full length N-protein into segments of about 5-150, 7-130, 8-110, 10-100, 10-90, 10-80, 10-70, 10-75, 10-60, or 10-50 amino acid residues provide peptide antigens for use in the methods. Other examples of peptide antigens include full length SARS CoV-2 S-protein, at least one specific epitope of full length SARS CoV-2 S-protein based on sequence and/or immunogenicity analysis represented by respective peptides. Peptides represented by SARS CoV-2 S-protein epitope map based on segmenting full length S-protein in segments of about 5-150, 7-130, 8-110, 10-100, 10-90, 10-80, 10-70, 10-75, 10-60, or 10-50 amino acid residues provide peptide antigens for use in the methods.

Particular peptide antigens related to SARS CoV-2 peptide antigens proteins are presented in Table 1. These peptides may comprise antigens and/or epitopes for human memory T cell surface receptors that are specific for SARS CoV-2 and can accordingly be used as components in the methods, devices and kits described herein for identification of such memory T cells.

“Indicator compound” refers to a substance that indicates the level of nucleotide in a sample. For example, indicator compounds include dyes that label RNA, DNA or both. In some instances, indicator compounds comprise fluorescent nucleotide dyes that exhibit excitation and emission wavelengths that are not blocked or absorbed by red blood cells.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Methods

In a first embodiment, methods to ascertain prior exposure of a subject to an infectious pathogen are provided. Prior exposure of a subject to a pathogen could occur by natural exposure or by vaccination against said pathogen. In some embodiments the pathogen may a virus such as a syncytial virus or a human corona virus. In some embodiments the pathogen is SARS CoV-2 virus. In other embodiments, the pathogen is a bacterial pathogen, such as a Borrelia pathogen for Lyme disease.

In some embodiments, the methods described herein include obtaining a sample, such as blood from a subject. For example, blood may be drawn from a subject via finger prick or venipuncture. In some embodiments, the volume of blood drawn is sufficient for analysis of the memory T cells comprised therein. For example, blood samples of certain embodiments may comprise at least about 1.0 mL to about 10 mL of liquid whole blood. For example, blood samples of particular embodiments may comprise about 1.0 mL, about 2.0 mL, about 3.0 mL, about 4.0 mL, about 5.0 mL, about 6.0 mL, about 7.0 mL, about 8.0 mL, about 9.0 mL, or about 10.0 mL of whole blood. In some embodiments, the blood is collected into a container comprising appropriate storage components, buffers and preservatives, including tubes that comprise heparin as an anti-coagulant.

In some embodiments, blood samples are centrifuged in order to separate the whole blood into separate layers comprising plasma, red blood cells (RBCs) and lymphocytes. In some embodiments, the memory T cells are present in a band that forms, upon centrifugation, between the plasma and the RBCs. This band comprises the lymphocytes, such as memory T cells, from the whole blood sample, and is known as the “buffy coat.”

In some embodiments, after a whole blood sample is centrifuged to separate the plasma, RBC and buffy coat layers, about 30 μL to about 100 μL of the buffy coat layer comprising lymphocytes, such as memory T cells, is removed. For example, particular embodiments may comprise removal of about 30 μL, about 40 μL, about 50 μL, about 60 μL, about 70 μL, about 80 μL, about 90 μL, or about 100 μL of the buffy coat layer.

In some embodiments, approximately half of the removed buffy coat layer is added to a first well of a multi-well plate, such as a 96 or 384 well plate. For example, particular embodiments may comprise adding about 15 μL, about 20 μL, about 25 μL, about 30 μL, about 35 μL, about 40 μL, about 45 μL, or about 50 μL of the buffy coat layer to a first well of a multi-well plate. In some aspects, the buffy coat sample volume added to the first well of a multi-well plate comprises the test sample.

In some embodiments, the remaining half of the buffy coat layer is added to a second well of a multi-well plate, such as a 96 or 384 well plate. For example, particular embodiments may comprise adding about 15 μL, about 20 μL, about 25 μL, about 30 μL, about 35 μL, about 40 μL, about 45 μL, or about 50 μL of the buffy coat layer to a second well of a multi-well plate. In some aspects, the buffy coat sample volume added to the second well of a multi-well plate comprises the control sample.

In some embodiments, an appropriate volume of test well solution is added to the test sample in the first well of the multi-well plate. In some embodiments, the test well solution comprises specific peptide antigens, such as SARS CoV-2 peptide antigens, corresponding to surface receptors present on the memory T cell of interest, i.e. memory T cells expressing surface receptors capable of binding to the peptide antigens of interest. In some embodiments the test well solution including the specific peptide antigens of interest, also comprises a balanced salt solution, a buffer, and an energy source, such as glucose.

In some embodiments, the test well solution also includes a predetermined concentration of an indicator compound, such as a nucleotide dye, i.e., fluorescent RNA and/or DNA dyes. In some embodiments, the nucleotide dye is added simultaneously with the test sample solution. In other embodiments, the nucleotide dye is added sequentially, before or after addition of the test well solution. In some embodiments, the nucleotide dyes provide penetration of cell and nuclear membranes and bind to all DNA, RNA, or both DNA and RNA molecules, without impacting cell function. In some embodiments, the nucleotide dyes comprise fluorescent dyes with excitation and emission wavelengths that are not absorbed and/or blocked by RBCs. For example, has an excitation between about 330-360 nm and an emission at greater than between about 500-600 nm.

In some embodiments, from about 50 μL to about 150 μL of test well solution is added to the test sample in the first well of the multi-well plate. For example, in some embodiments, about 50 μL, about 60 μL, about 70 μL, about 80 μL, about 90 μL, about 100 μL, about 110 μL, about 120 μL, about 130 μL, about 140 μL, or about 150 μL of test well solution is added to the test sample in the first well of the multi-well plate.

In some embodiments, an appropriate volume of control well solution is added to the control sample in the second well of the multi-well plate. In some embodiments, the control well solution comprises all of the same components as the test well solution except it lacks the specific peptide antigens, such as SARS CoV-2 peptide antigens. For example, in some embodiments the control well solution comprises a balanced salt solution, a buffer, and an energy source, such as glucose.

In some embodiments, the control well solution also includes a predetermined concentration of an indicator compound, such as a nucleotide dye, i.e., fluorescent RNA and/or DNA dyes. In some embodiments, the nucleotide dye is added simultaneously with the control sample solution. In other embodiments, the nucleotide dye is added sequentially, before or after addition of the control well solution.

In some embodiments, from about 50 μL to about 150 μL of control well solution is added to the control sample in the second well of the multi-well plate. For example, in some embodiments, about 50 μL, about 60 μL, about 70 μL, about 80 μL, about 90 μL, about 100 μL, about 110 μL, about 120 μL, about 130 μL, about 140 μL, or about 150 μL of control well solution is added to the control sample in the second well of the multi-well plate.

In some embodiments, the test and control samples comprising the buffy coat and the additional test and control sample solutions are incubated for about 10 minutes to about 60 minutes, at a temperature of about 25° C. to about 40° C. For example, in some embodiments, the test and control samples are incubated for about 10 minutes, about 20 minutes, about 30 minutes, about 40 minutes or about 50 minutes; at a temperature of about 25° C., about 30° C., about 35° C., or about 40° C. In some embodiments, the test and control samples are incubated at about 37° C., for about 30 minutes. In some embodiments, the test and control samples may be incubated at about 37° C. for longer periods, such as about 2 hours, about 4 hours, about 6 hours, about 12 hours, about 18 hours or about 24 hours prior to reading the test result.

In some embodiments, after incubation, the first well comprising the test sample and the second well comprising the control sample are analyzed to ascertain the total amount of labeled nucleotide present in each well. In some embodiments, the analysis may comprise visual inspection. In other embodiments, the analysis may comprise analysis by a plate reader. In other embodiment, analysis may include fluorescence analysis such as scanning of the test well and control well with a fluorometer. In other embodiments, analysis may include spectroscopic analysis of test and control samples using electromagnetic radiation, such as, without limitation, absorption spectroscopy (ultra-violet, visible, or infrared), including reflectance or transmittance spectroscopy, or emission spectroscopy, including fluorescence and luminescence spectroscopy, Raman spectroscopy, and any type of radiation scattering.

In one embodiment, the indicator compound is a fluorescent compound, such as a fluorescent dye, that has an excitation at between about 300-400 nm, or between about 320-380 nm, or between 330-360 nm, and emission at greater than about 600 nm, or greater than 600 nm and less than about 1000 nm.

In some embodiments, after analysis of the test and control samples is complete, the amount of nucleotide present in each sample is compared. If the test sample exhibits higher nucleotide expression, this indicates that the test sample, i.e., the buffy coat sample, includes memory T cells expressing surface receptors specific for the peptide antigen, such as SARS CoV-2 peptide antigens, that was present in the test well solution. Specifically, the surface receptors present on the memory T cells from the buffy coat sample interacted with the peptide antigens in the test well solution causing memory T cell transformation which is characterized increased RNA and/or DNA expression for increased immune effector protein expression and whole genome replication for rapid cell division.

Accordingly, if the test sample expresses increased RNA and/or DNA expression compared to the control sample, this indicates that the sample is from and individual who has experienced previous exposure, vaccination, and/or infection with the pathogen, such as SARS CoV-2 virus, associated with the peptide antigen, such as SARS CoV-2 peptide antigens, present in the test well solution.

Also, if the test sample and control samples express the same level of RNA and/or DNA expression, this indicates that the that the sample is from and individual who has not experienced previous exposure, vaccination, and/or infection with the pathogen associated with the peptide antigens present in the test well solution.

### A. Peptide Antigens

In some aspects, the peptide antigens comprise a protein or peptide that binds to specific receptors present on the cell surface of a particular population of memory T cells. Peptide antigens may include any antigenic peptide or protein from a pathogen of interest. In some embodiments, the peptide antigens are antigens from particular bacterial and/or viral pathogens.

In some embodiments, the peptide antigens are highly specific bacterial and/or viral peptide antigens. In some embodiments, the bacterial and/or viral peptide antigens are specific for an antigen of interest, and have little to no measurable cross reactivity with related bacterial or viral antigens. For example, seasonal coronavirus infection leads to a mild cold or flu like illness to which many people have prior exposure. Such individuals express memory T cells corresponding to seasonal cold and flu coronavirus strains. In an embodiment, the peptide antigens used in the methods have little or no binding for seasonal cold and/or flu (influenza a and/or b) coronavirus strains.

Accordingly, the peptide antigens for use in the methods described herein are specific to memory T cells for particular infectious agents, such as SARS CoV-2, RSV, and/or Lyme disease. Therefore, the peptide antigens exhibit little, if any, non-specific cross reactivity with memory T cells specific for seasonal colds and flus, and will only react with the appropriate, specific memory T cells of interest, such as SARS CoV-2 memory T cells, Lyme disease specific T cells, and RSV specific memory T cells, for example.

In some embodiments, the methods provide multiple antigen peptides specific for SARS CoV-2 that do not cross react to any of the seasonal coronavirus subtypes in general/season cold and flu circulation. In some embodiments, the methods comprise at least three to about fifteen different peptide antigens that are specific to SARS CoV-2 virus without exhibiting cross reactivity with seasonal coronavirus strains.

In certain embodiments, the peptide antigens comprise SARS CoV-2 antigen peptide sequences for memory T cell assays are based on Nucleocapsid (N), Spike (S), and M protein sequences as provided in Table 1. In some embodiments, the antigenic peptides are chemically synthesized with an N-Terminal Biotin and miniPEG linker.

### B. Indicator Compounds

In some embodiments, the methods provide specific and sensitive indicator compounds for detecting and indicating the level of nucleotide, such as RNA and/or DNA, expression in a given sample. In some embodiments, the indicator compounds comprise several different classes of fluorescent dyes. In some embodiments, indicator compounds bind only to RNA. In other embodiments, indicator compounds may bind only to DNA. In still other embodiments, indicator compounds my bind both RNA and DNA.

In some embodiments, indicator compounds are fluorescent compounds. In some embodiments, the fluorescent indicator compounds exhibit at least about 15 to about 20 times greater fluorescent signal upon nucleotide binding as compared to unbound indicator.

In some embodiments, the indicator compounds are able to rapidly diffuse through both the cellular and nuclear membranes. In some embodiments, the indicator compounds are non-toxic and do not affect cellular processed upon binding to nucleic acid.

In some aspects, the currently technology provides the use of separate RNA specific indicator compounds in conjunction with certain DNA specific indicator compounds in order to ascertain the RNA and DNA expression separately. In some embodiments, the ratio of RNA expression to DNA expression may provide a useful calculation related to determination of assay results, such as the amplitude, timing and sequencing of the memory T cell transformation response to antigen.

In some aspects, the indicator compounds exhibit distinct excitation and emission wavelength(s) for specific dyes used for separate analysis of RNA and DNA, so that RNA and DNA may be analyzed separately based on the distinct excitation and emission wavelengths of the respective indicator compounds. In other embodiments, the current technology provides the same indicator compound for analysis of both RNA and DNA.

In some aspects, the indicator compound can be a fluorescent compound, dye, or stain selective for RNA. For example, the indicator can be a cell permeant nucleic acid stain that selectively stains intracellular RNA, such as SYTO™ 13 Green fluorescent nucleic acid stain. In some embodiments, the stain is essentially non-fluorescent in the absence of nucleic acids, and exhibits bright green fluorescence when bound to RNA. In some embodiments the indicator compound exhibits an absorption/emission maxima of about 490 nm to about 530 nm. In some embodiments, the indicator exhibits a strong signal when bound to RNA and has a weak fluorescent signal when bound to DNA.

In another aspect, the indicator compound can be a fluorescent compound, dye, or stain non-selective for RNA or DNA, but is capable of staining both RNA and DNA. Examples cell-permeant fluorescent nucleic acid stains that exhibit fluorescence upon binding to nucleic acids, such as those sold under the trade name SYTO™. Another exemplary indicator compound is a dye compound that is non-toxic to cells and non-toxic to nucleic acid, such as Hoechst stains identified as Hoechst stain 33342 and 34580. Both stains are excited by ultraviolet light at around 350 nm and both emit a blue-cyan fluorescent light around 461 nm. The Stokes shift between the excitation and emission spectra of around 100 nm is beneficial. These dyes bind the minor groove of double stranded DNA.

In another embodiment, the method utilizes two indicator compounds, a first indicator compound that selectively stains RNA or DNA and a second indicator compound that non-specifically stains RNA and DNA. In one embodiment, the indicator compound or compounds have an absorption at between about 300-400 nm, about 320-380 nm, or about 330-360 nm, and an emission at greater than about 500 nm to about 600 nm. In embodiments where two indicator compounds are used, a first indicator compound has a first absorption/emission profile that is different from a second absorption/emission profile of the second indicator compound.

### III. Devices and Kits

Severe acute respiratory syndrome coronavirus 2 (SARS CoV-2) is the virus strain that causes coronavirus disease 2019 (COVID-19), a respiratory illness. The methods described herein provide a sensitive and specific method for the detection of memory T cells specific for particular pathogenic peptide antigens, such as memory T cells specific for SARS CoV-2 viral peptide antigens. The methods provide for ascertaining whether an individual has been previously exposed to, or infected with SARS CoV-2. In addition, in some embodiments, the methods, kits and devices provided herein provide valuable knowledge related to whether an individual is protected from future infections, such as SARS CoV-2 infection, via an immune response conferred by SARS CoV-2 specific memory T cells. Accordingly, high volume and rapid screening provided by the methods, devices and kits provided herein assay for subjects that were previously infected by SARS CoV-2 provides critical epidemiological data concerning the COVID-19 status of each subject and the population status by region concerning the spread of the virus. Devices and kits for performing one or more of the methods provided herein, along with instructions for using the devices and kits in the provided methods of detecting memory T cells specific for particular pathogens, such as SARS CoV-2 virus are also provided.

### IV. EXAMPLES

The following examples are illustrative in nature and are in no way intended to be limiting.

### Example 1

### Method for Detecting SARS CoV-2 Specific Memory T Cells

Five to 10 mL of blood is drawn from a subject via venipuncture and placed into a tube containing heparin as an anticoagulant. The tube is centrifuged to separate RBCs from plasma. During centrifugation, lymphocytes form a buffy coat band between the red blood cells and plasma. 100 μL of buffy coat cells are removed from the plasma/RBC interface of the centrifuged sample tube. Half of the buffy coat volume (50 μL) is added to one well of a 96 well plate to produce a test well. The other half of the buffy coat sample is added to a second well to produce a control well.

100 μL test solution containing SARS CoV-2 specific peptide antigens, a balanced salt solution, a buffer, glucose as an energy source, and a predetermined concentration of a fluorescent nucleotide dye is added to the test well.

100 μL control solution containing a balanced salt solution, a buffer, glucose as an energy source, and a predetermined concentration of a fluorescent nucleotide dye is added to the control well. Control solution is same as test solution without the SARS CoV-2 specific peptide antigens.

The 96 well plate is incubated at 37° C. for 30 minutes. After incubation, the two wells are scanned using a fluorometer and the signal of the two wells is compared. The signal from the test well is higher than the control well indicating that the buffy coat sample is positive for the presence of memory T cells specific for SARS CoV-2 virus peptide antigens. This indicates the subject had prior exposure to SARS CoV-2 virus, resulting in memory T cells being activated and rapidly generating new RNA & DNA in the test leading to an increase in fluorescent signal in the test sample as compared to the control sample.

### Example 2

### Point of Care Testing for SARS CoV-2 Specific Memory T Cells

50 μl of blood is drawn from a subject via finger prick and placed into a tube containing heparin as an anticoagulant. The tube is centrifuged to separate RBCs from plasma. During centrifugation, lymphocytes form a buffy coat band between the red blood cells and plasma. 5 μL of buffy coat cells are removed from the plasma/RBC interface of the centrifuged sample tube. Half of the buffy coat volume (2.5 μL) is added to one well of a 96 well plate to produce a test well. The other half of the buffy coat sample is added to a second well to produce a control well.

5 μL test solution containing SARS CoV-2 specific peptide antigens, a balanced salt solution, a buffer, glucose as an energy source, and a predetermined concentration of a fluorescent nucleotide dye is added to the test well.

5 μL control solution containing a balanced salt solution, a buffer, glucose as an energy source, and a predetermined concentration of a fluorescent nucleotide dye is added to the control well. Control solution is same as test solution without the SARS CoV-2 specific peptide antigens.

The 96 well plate is incubated at 37° C. for 10 minutes. After incubation, the two wells are scanned using a fluorometer and the signal of the two wells is compared. The signal from the test well is higher than the control well indicating that the buffy coat sample is positive for the presence of memory T cells specific for SARS CoV-2 virus peptide antigens. This indicates the subject had prior exposure to SARS CoV-2 virus, resulting in memory T cells being activated and rapidly generating new RNA and DNA in the test leading to an increase in fluorescent signal in the test sample as compared to the control sample.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method to identify presence of SARS CoV-2-specific T cells in a sample from a subject to ascertain prior exposure to, or vaccination with, SARS CoV-2 virus, comprising:
exposing a biological sample comprising memory T-cells from the subject to one or more peptide antigens specific for SARS CoV-2;
contacting the exposed memory T-cells with an indicator compound that associates with RNA, DNA, or both, wherein the contacting is simultaneous with or sequential to the exposing;
analyzing the memory T-cells for indicator compound.

2. The method of claim 1, where the biological sample is a blood sample or a fraction thereof.

3. (canceled)

4. The method of claim 2, where the biological sample is a fraction of the blood sample, wherein the fraction is the buffy coat fraction or peripheral blood mononuclear cells (PBMCs) or a mixture of buffy coat fraction and PBMCs.

5. The method of claim 1, wherein said exposing to one or more peptide antigens specific for SARS CoV-2 comprises exposing to a solution comprising the one or more peptide antigens, the indicator compound and one or more of a buffer, an energy source for the cells, and a balanced salt solution, thereby simultaneously with said exposing, contacting the T cells with the indicator compound.

6. The method of claim 1, wherein the method further comprises exposing a second biological sample comprising memory T-cells to a control reagent that (i) lacks the one or more peptide antigens specific for SARS CoV-2 and (ii) comprises a control indicator compound that associates with RNA, DNA, or both, to thereby generate a control sample.

7. The method of claim 6, wherein the second biological sample is from a subject, and wherein the biological sample is from the same subject or wherein the second biological sample is a portion of the biological sample.

8. The method of claim 6, wherein the indicator compound and the control indicator compound are the same.

9. The method of claim 6, wherein said analyzing comprises measuring signal of indicator compound associated with memory T-cells in the biological sample and measuring signal of control indicator compound associated with memory T-cells in the control sample.

10. The method of claim 6, wherein said analyzing comprises measuring signal of indicator compound associated with RNA in the biological sample and measuring signal of control indicator compound associated with RNA in the second biological sample.

11. The method of claim 1, wherein said analyzing comprises measuring an RNA signal based on signal of indicator compound associated with RNA, measuring a DNA signal based on signal of indicator compound associated with DNA, and determining a ratio of RNA signal to DNA signal or of DNA signal to RNA signal.

12. The method of claim 1, wherein the indicator compound is a fluorescent dye that selectively stains RNA.

13. The method of claim 1, wherein said contacting comprises contacting the exposed memory T-cells with a first indicator compound that selectively stains RNA or DNA and with a second indicator compound that non-specifically stains RNA and DNA.

14. The method of claim 1, wherein the indicator compound has an excitation between about 330-360 nm and an emission at greater than between about 500-600 nm.

15. The method of claim 1, wherein the memory T-cells are CD4+ and/or CD8+ T cells.

16. The method of claim 1, further comprising after said contacting and before said analyzing, incubating for a period of time.

17. The method of claim 16, wherein said incubating is at a temperature of between about 25-40° C.

18. The method of claim 16, wherein said period of time is between about 10-60 minutes or between about 10-30 minutes.

19. The method of claim 1, wherein the one or more peptide antigens specific for SARS CoV-2 comprises between 2-20 peptide antigens specific for SARS CoV-2 or between 3-15 peptide antigens specific for SARS CoV-2.

20. The method of claim 1, wherein exposing further comprising exposing the biological sample to one or more peptide antigens non-specific for SARS CoV-2.

21. The method of claim 1, wherein the one or more peptide antigens specific for SARS CoV-2 comprise one or more of the peptides identified as SEQ ID NO: 1-SEQ ID NO: 12.

22. A method to ascertain prior exposure of a subject to an infectious pathogen, comprising:
exposing a biological sample comprising memory T-cells from the subject to one or more peptide antigens specific for the pathogen;
contacting the exposed memory T-cells with an indicator compound that associates with RNA, DNA, or both, wherein the contacting is simultaneous with or sequential to the exposing;
analyzing the memory T-cells for indicator compound.

23-27. (canceled)

28. The method of claim 22, wherein said exposing to one or more peptide antigens specific for the pathogen comprises exposing to a solution comprising the one or more peptide antigens, the indicator compound and one or more of a buffer, an energy source for the cells, and a balanced salt solution, thereby simultaneously with said exposing, contacting the T cells with the indicator compound.

29. The method of claim 22, wherein the method further comprises exposing a second biological sample comprising memory T-cells to a control reagent that (i) lacks the one or more peptide antigens specific for the pathogen and (ii) comprises a control indicator compound that associates with RNA, DNA, or both, to thereby generate a control sample.

30-41. (canceled)

42. The method of claim 22, wherein the one or more peptide antigens specific for the pathogen comprises between 2-20 peptide antigens specific for the pathogen or between 3-15 peptide antigens specific for the pathogen.

43. The method of claim 22, wherein exposing further comprising exposing the biological sample to one or more peptide antigens non-specific for the pathogen.

44. A method to identify presence of infectious agent-specific T cells in a sample from a subject to ascertain prior exposure to, or vaccination with, the infectious agent, comprising:
exposing a biological sample comprising memory T-cells from the subject to one or more peptide antigens specific for an infectious agent;
contacting the exposed memory T-cells with an indicator compound that associates with RNA, DNA, or both, wherein the contacting is simultaneous with or sequential to the exposing;
analyzing the memory T-cells for indicator compound.
